(19) United States
(12) Patent Application Publication
(10) Pub. No.: US 2026/0014251 A1
(43) Pub. Date: Jan. 15, 2026

(54) COMPOSITIONS, METHODS, AND USES FOR POLYNUCLEOTIDE FORMULATIONS FOR DRYING AND PROLONGED STORAGE

(71) Applicant: VitriVax, Inc., Boulder, CO (US)

(72) Inventors: Lorena Napolitano, Boulder, CO (US); Emma Palm, Boulder, CO (US); Marina Barreto Felisbino, Boulder, CO (US); Joseph Spencer, II, Boulder, CO (US)

(21) Appl. No.: 19/273,245

(22) Filed: Jul. 18, 2025

Related U.S. Application Data
(63) Continuation of application No. PCT/US2024/012635, filed on Jan. 23, 2024.
(60) Provisional application No. 63/529,599, filed on Jul. 28, 2023.

(51) Int. Cl. A61K 39/385; A61K 9/51; A61K 31/7105; A61K 39/00; A61K 39/02; A61K 39/08; A61K 39/12; A61K 39/125; A61K 39/13; A61K 39/145; A61K 39/155; A61K 39/165; A61K 39/175; A61K 39/205; A61K 39/21; A61K 39/215; A61K 39/235; A61K 39/245; A61K 39/25; A61K 39/29

(52) U.S. Cl. CPC A61K 39/385; A61K 9/5123; A61K 31/7105; A61K 39/0002; A61K 39/0208; A61K 39/0225; A61K 39/0291; A61K 39/08; A61K 39/099; A61K 39/12; A61K 39/125; A61K 39/13; A61K 39/145; A61K 39/155; A61K 39/165; A61K 39/175; A61K 39/205; A61K 39/21; A61K 39/215; A61K 39/235; A61K 39/245; A61K 39/25; A61K 39/29; A61K 39/292; A61K 2039/53

(57) ABSTRACT

Embodiments of the present disclosure provide novel compositions and methods for making and using thermostable polynucleotide-containing formulations. In certain embodiments, compositions and methods are disclosed for creating thermostable polynucleotides and/or thermostable polynucleotides encoding at least one therapeutic agent for use in therapies for the treatment of health conditions in a subject. In some embodiments, compositions and methods are disclosed for creating thermostable polynucleotides for use in therapeutics, vaccines, and targeted gene therapies. In other embodiments, compositions and methods are disclosed for creating thermostable polynucleotides capable of being coated or encased for prolonged storage and/or timed-delivery.

### CROSS-REFERENCED APPLICATIONS

This Continuation Application claims priority to International Application PCT/US2024/012635 filed January 23, 2024, which claims priority to U.S. Provisional Application No. 63/529,599 filed July 28, 2023. These applications are incorporated herein by reference in their entireties for all purposes.

### FIELD

Embodiments of the present disclosure provide novel compositions and methods for making and using thermostable polynucleotide-containing formulations. In certain embodiments, compositions and methods are disclosed for creating thermostable polynucleotides for use in therapies for the treatment or prevention of health conditions in a subject. In some embodiments, compositions and methods are disclosed for creating thermostable polynucleotides for use in vaccines and gene therapies. In other embodiments, compositions and methods are disclosed for creating thermostable polynucleotides capable of being coated or encased by coating layers for prolonged storage and/or timed-delivery.

### BACKGROUND

Therapeutic impacts of vaccines and the transfer of polynucleotides in therapeutic settings can be compromised by many challenges. One challenge is the delivery of therapeutically relevant polynucleotides to a subject. For example, multiple administrations are often required to provide successful immunity to certain pathogens, which can decrease the likelihood that a subject will take the necessary steps to obtain a required second or more administrations. In addition, polynucleotide formulations often exhibit increased instability during elevated temperatures, during storage, transport, and handling. Supply chains for polynucleotide therapies such as vaccines can require expensive and logistically complex refrigeration (e.g., cold chain requirements), limiting availability. Additionally, polynucleotide formulations are typically associated with high manufacturing costs, which can limit their availability to those in need and when demand is high (e.g., a viral outbreak).

### SUMMARY

Embodiments of the present disclosure provide novel compositions and methods for making and using thermostable polynucleotides and polynucleotide complex formulations for prolonged storage at elevated temperatures, improved and more accurate therapeutic administration, and/or timed delivery. In certain embodiments, one or more polynucleotide construct or complex thereof or modified polynucleotide construct or complex thereof can include, but is not limited to, a polynucleotide or modified polynucleotide construct or complex thereof encoding at least one therapeutic agent or antigen, or wherein the polynucleotide or modified polynucleotide construct or complex thereof is therapeutically relevant in absence of encoding a secondary molecule. In accordance with these embodiments, the one or more polynucleotide construct or complex thereof or modified polynucleotide construct or complex thereof encoding at least one therapeutic agent can include, but is not limited to, any form of one or more of DNA, RNA, or a mixture or complex thereof. In certain embodiments, one or more polynucleotide construct or complex thereof or modified polynucleotide construct or complex thereof can include, but is not limited to, a polynucleotide or complex thereof encoding one or therapeutic agents including, but not limited to, one or more antigens or immunogenic agent; or includes at least one polynucleotide capable of inducing an immune response in a subject or has immunomodulatory properties when introduced to a subject.

In certain embodiments, compositions disclosed herein can include an aqueous or liquid composition including, but not limited to, a polynucleotide construct or complex thereof or modified polynucleotide construct or complex thereof encoding one or more therapeutic agents, and one or more non-reducing disaccharide agents, where the aqueous composition does not include an unrelated agent where an unrelated agent includes an adjuvant capable of inducing a general immune response in a subject. In certain embodiments, compositions disclosed herein can include an aqueous or liquid composition including, but not limited to, a polynucleotide construct or complex thereof or modified polynucleotide construct or complex thereof encoding one or more therapeutic agents, one or more non-reducing disaccharide agents; and one or more polymers, where the aqueous composition does not include an unrelated agent where an unrelated agent includes an adjuvant capable of inducing a general immune response in a subject. In some embodiments, compositions disclosed herein can include, but are not limited to, a single lipid nanoparticle or a mixture of lipid nanoparticles (LNPs) for encapsulating the polynucleotide construct or complex thereof or modified polynucleotide construct or complex thereof, or a polynucleotide construct or complex thereof or modified polynucleotide construct or complex thereof encoding one or more therapeutic agents (e.g., polypeptide or chimera). As disclosed herein, an LNP is composed of cationic or ionizable lipids, sterols (e.g., cholesterol), phospholipids, and PEG-anchored lipids, for example, LNPs and LNP components are understood by one of skill in the art. In other embodiments, compositions disclosed herein can include one or more of a volatile salts or volatile buffer. In certain embodiments, compositions disclosed herein specifically exclude one or more of a volatile salts or volatile buffer.

In other embodiments, essentially dry (e.g., post spray-dried) formulations containing at least one polynucleotide construct or complex thereof or modified polynucleotide construct or complex thereof or a polynucleotide construct or complex thereof or modified polynucleotide construct or complex thereof encoding one or more therapeutic agents can be encapsulated or coated with one or more coating layers to produce coated or encapsulated polynucleotide constructs or complexes thereof or modified polynucleotide constructs or complexes thereof microparticles. In certain embodiments, the coated or encapsulated microparticles can be for a timed-release delivery of the polynucleotide construct or complex thereof or modified polynucleotide construct or complex thereof. In some embodiments, primary and boost doses of polynucleotide construct or complex thereof or modified polynucleotide construct or complex thereof-containing microparticles can be administered to a subject in a single administration. In other embodiments, polynucleotide construct or complex thereof or modified polynucleotide construct or complex thereof-containing microparticles can include immunogenic agents against one or more pathogens either in the same or in separate microparticles. In accordance with these embodiments, 1, 2, 3, 4, 5, up to 10, up to 20, up to 30, up to 40, up to 100, up to 200, up to 300, up to 400, or any number in between or more than 400 coating layers can encase the microparticles where the coating layers dissolve or dissolve at a predetermined rate in a subject once administered, to expose the polynucleotide construct or complex thereof or modified polynucleotide construct or complex thereof-containing microparticles to the subject to reduce onset, prevent and/or treat a health condition in the subject.

In other embodiments, the coated or encased polynucleotide construct or complex thereof or modified polynucleotide construct or complex thereof-containing microparticles can increase stability and/or compatibility of the polynucleotide construct or complex thereof or modified polynucleotide construct or complex thereof. In certain embodiments, polynucleotide constructs or complexes thereof encoding incompatible polypeptides or other molecules can be physically separated in different coating layers of encapsulated microparticles; for example, where a core harbors a first polynucleotide construct or complex thereof encoding a first polypeptide and another, separate layer of the same microparticles can harbor a second polynucleotide construct or complex thereof encoding a second polypeptide without compatibility issue and for timed-release of each of the second and first polynucleotide construct or complex thereof. In certain embodiments, the coating layers of microparticles disclosed herein are applied by atomic layer deposition (e.g., metalloorganic compositions such as metal oxide or metal alkoxide or the like).

In other embodiments, pharmaceutical composition are contemplated of use herein that include a reconstituted composition of coated or uncoated essentially dried thermostable formulation containing at least one polynucleotide construct or complex thereof, and a pharmaceutically acceptable excipient. In certain embodiments, a pharmaceutical composition contemplated herein can be used as a vaccine of use against one or more pathogens or against cancer, or in delivery of a polynucleotide-containing formulation for gene therapy to treat or cure a health condition in a subject.

Other embodiments include methods for preparing a composition disclosed herein. In certain embodiments, methods can include combining the components of a formulation disclosed herein to create a liquid formulation including, but not limited to, at least one polynucleotide construct or complex thereof or modified polynucleotide construct or complex thereof, or at least one polynucleotide construct or complex thereof or modified polynucleotide construct or complex thereof encoding one or more therapeutic agents to create a liquid formulation; at least one polymer, at least one disaccharide, and at least one of: atomizing the liquid formulation in a gas stream to create particulates and spray-drying the liquid formulation to create an essentially dry formulation. In some embodiments, at least 1.0% of the polynucleotide construct or complex thereof or modified polynucleotide construct or complex thereof encoding one or more therapeutic agents is stable or wherein less than 99.0%, or less than 80%, or less than 70.0% or less than 60% or less than 50% or less than 40% of the polynucleotide construct or complex thereof or modified polynucleotide construct or complex thereof encoding one or more therapeutic agents is degraded during and after this process.

In some embodiments, the at least one polynucleotide construct or complex thereof can encode at least one therapeutic agent including an antigen (e.g., an immunogenic antigen derived from a pathogenic organism). In accordance with these embodiments, the at least one antigen can include, but is not limited to, at least one viral antigen, bacterial antigen, fungal antigen, protozoan-derived agent, toxin, fragment thereof, or a combination thereof. In some embodiments, at least one polynucleotide construct or complex thereof or modified polynucleotide construct or complex thereof can further encode at least on other agent including, but not limited to, at least one polypeptide, two or more different polynucleotides, a capsomere, one or more lipids, one or more lipid nanoparticles (LNPs), or other polynucleotide complexable agents or the like.

In some embodiments, the liquid formulation containing one or more polynucleotide construct or complex thereof or modified polynucleotide construct or complex thereof further includes, but is not limited to, one or more polymers including, but not limited to, polyvinyl alcohol (PVA), polyacrylic acid (PAA), polyethylene glycol (PEG), povidone, polymethacrylate-based copolymers (e.g., Eudragit), other medium to high molecular weight polymers or combinations thereof.

In certain embodiments, methods for eliciting a therapeutic response, or an immune response against one or more pathogenic organisms or stimulate an immune response against cancer or other antigens in a subject are provided. In accordance with these embodiments, a pharmaceutical composition disclosed herein can be administered to a subject, where the polynucleotide construct or complex thereof or modified polynucleotide construct or complex thereof, or the polynucleotide construct or complex thereof or modified polynucleotide construct or complex thereof encoding one or more therapeutic agents is introduced to a subject as uncoated microparticles or coated microparticles for timed-release. In certain embodiments, the polynucleotide construct or complex thereof or modified polynucleotide construct or complex thereof encodes one or more immunogenic agent derived from at least one pathogenic organism, cancer, or other antigen, and elicits an immune response in the subject to the one or more immunogenic agents at the time of introduction to the subject in uncoated microparticles or at a later time based on the number of ALD-applied coating layers for a timed-release exposure or multiple exposures from the same or different coated microparticles disclosed herein.

Yet other embodiments, disclose kits for storing, transporting or delivering at least one composition or pharmaceutical composition disclosed herein, and at least one container.

### DETAILED DESCRIPTION

In the following sections, various exemplary compositions and methods are described in order to detail various embodiments. It will be obvious to one skilled in the art that practicing the various embodiments does not require the employment of all or even some of the specific details outlined herein, but rather that concentrations, times and other specific details may be modified through routine experimentation. In some embodiments, well known methods or components have not been included in the description.

Embodiments of the present disclosure provide novel compositions and methods for making and using thermostable polynucleotides and polynucleotide complex formulations for prolonged storage at elevated temperatures, improved and more accurate therapeutic administration, and/or timed delivery. In some embodiments, these formulations are essentially dried to form a stable compositions and/or encapsulated or coated using ALD. In certain embodiments, stabilized formulations disclosed herein can be in the form of microparticles and further encased or coated with at least one coating layer for complete encapsulation of the microparticles. In some embodiments, the at least one coating layer can include metalloorganic compositions applied by ALD of the stabilized, essentially dry microparticles disclosed herein. In certain embodiments, compositions disclosed herein can include an aqueous or liquid composition including, but not limited to, a polynucleotide construct or complex thereof or modified polynucleotide construct or complex thereof encoding one or more therapeutic agents, and one or more non-reducing disaccharide agents, where the aqueous composition does not include an unrelated agent where an unrelated agent includes an adjuvant capable of inducing a general immune response in a subject. In certain embodiments, one or more polynucleotide construct or complex thereof or modified polynucleotide construct or complex thereof can include, but is not limited to, a polynucleotide or modified polynucleotide construct or complex thereof encoding at least one therapeutic agent or antigen, or wherein the polynucleotide or modified polynucleotide construct or complex thereof is therapeutically relevant in absence of encoding a secondary molecule and in the presence of at least one polymer. In accordance with these embodiments, the one or more polynucleotide construct or complex thereof or modified polynucleotide construct or complex thereof encoding at least one therapeutic agent can include, but is not limited to, any form of one or more of DNA, RNA, or a mixture or complex thereof. In certain embodiments, one or more polynucleotide construct or complex thereof or modified polynucleotide construct or complex thereof can include, but is not limited to, a polynucleotide or complex thereof encoding one or therapeutic agents including, but not limited to, one or more antigens or immunogenic agent; or includes at least one polynucleotide capable of inducing an immune response in a subject or has immunomodulatory properties when introduced to a subject.

In some embodiments, the one or more polynucleotide construct or complex thereof or modified polynucleotide construct or complex thereof can include, but is not limited to, any form of one or more of DNA, siRNA, RNA (e.g., mRNA), or a mixture or complex thereof. In certain embodiments, the one or more polynucleotide construct or complex thereof or modified polynucleotide construct or complex thereof can include, but is not limited to, a polynucleotide or complex thereof encoding one or therapeutic agents and/or wherein the polynucleotide or modified polynucleotide construct or complex thereof is therapeutically relevant in absence of encoding a secondary molecule including, but not limited to, one or more antigens or immunogenic agent; or includes at least one polynucleotide capable of inducing an immune response in a subject or has immunomodulatory properties in a subject.

In certain embodiments, compositions disclosed herein include an aqueous composition including, but not limited to, a polynucleotide construct or complex thereof or modified polynucleotide construct or complex thereof encoding one or more therapeutic agents and/or wherein the polynucleotide or modified polynucleotide construct or complex thereof is therapeutically relevant in absence of encoding a secondary molecule, one or more non-reducing disaccharide agents; a neutral buffer (e.g., histidine, glycine, HEPES etc.), and one or more polymers, where the aqueous composition does not include an unrelated adjuvant wherein an unrelated adjuvant is an adjuvant capable of inducing a general and/or non-specific immune response in a subject. In some embodiments, compositions disclosed herein can include, but are not limited to, a single lipid nanoparticle or a mixture of lipid nanoparticles (LNPs) for encapsulating the polynucleotide construct or complex thereof or modified polynucleotide construct or complex thereof encoding one or more therapeutic agents. In other embodiments, aqueous compositions disclosed herein further include one or more of a salt, a volatile salts or volatile buffer. In other embodiments, aqueous compositions disclosed herein further specifically exclude a volatile salts or volatile buffer. In some embodiments, compositions prior to spray-drying can further include another salt. In accordance with this embodiment, the other salt can include sodium acetate; optionally, in trace amounts.

In some embodiments, the liquid formulation containing one or more polynucleotide construct or complex thereof or modified polynucleotide construct or complex thereof can include, but is not limited to, one or more non-reducing disaccharide. In accordance with these embodiments, the one or more disaccharides can include, but are not limited to, trehalose, sucrose, lactose, or other disaccharides or non-reducing disaccharides, or combinations thereof. In some embodiments disclosed herein, a neutral buffer can include, but is not limited to, histidine, glycine, HEPES, phosphate, citrate and the like or a combination thereof. In certain embodiments, a neutral buffer can include, histidine, glycine, HEPES or a combination thereof.

In some embodiments, the liquid formulation prior to forming stable essentially dry microparticles containing one or more polynucleotide construct or complex thereof or modified polynucleotide construct or complex thereof and/or one or more polynucleotide construct or complex thereof or modified polynucleotide construct or complex thereof encoding at least one therapeutic agent further includes, but is not limited to, one or more polymers including, but not limited to, polyvinyl alcohol (PVA), polyacrylic acid (PAA), polyethylene glycol (PEG), povidone, polymethacrylate-based copolymers (e.g., Eudragit), other medium to high molecular weight polymers, or low solubility polymers, or combinations thereof.

In some embodiments, the liquid formulation prior to forming stable essentially dry microparticles containing one or more polynucleotide construct or complex thereof or modified polynucleotide construct or complex thereof can include, but is not limited to, at least one additional agent. In certain embodiments, the at least one additional agent can include, but is not limited to, one or more of hydroxypropyl-beta-cyclodextrin, lysine, leucine, silk fibroin, inulin, alternative sugars comprising one or more of dextrose, maltodextrin, dextran, maltotriose, sorbitol, and mannitol. In certain embodiments, the at least one additional agent can include, but is not limited to, one or more agent capable of at least one of, preserving LNP integrity, reduce or prevent LNP leaking from a stabilized microparticle; for example, with or without ALD coating layers, LNPs and LNP components are understood by one of skill in the art. In certain embodiments, the at least one additional agents include, but is not limited to, one or more additional volatilizing agents, comprising an alcohol or the like; optionally, wherein the alcohol comprises methanol or ethanol or similar alcohol. In some embodiments, the at least one additional agent can include water. In accordance with these embodiments, the at least one additional agent can include water in absence of a volatile salt and/or volatile buffer.

In some embodiments, the liquid formulation prior to forming stable essentially dry microparticles containing one or more polynucleotide construct or complex thereof or modified polynucleotide construct or complex thereof and/or one or more polynucleotide construct or complex thereof or modified polynucleotide construct or complex thereof encoding at least one therapeutic agent-containing composition has minimal to no Tris buffer. In some embodiments, the liquid formulation prior to forming stable essentially dry microparticles containing one or more polynucleotide construct or complex thereof or modified polynucleotide construct or complex thereof and/or one or more polynucleotide construct or complex thereof or modified polynucleotide construct or complex thereof encoding at least one therapeutic agent-containing composition has no Tris buffer added to the liquid formulation in preparation for spray-drying. In other embodiments, the liquid formulation prior to forming stable essentially dry microparticles containing one or more polynucleotide construct or complex thereof or modified polynucleotide construct or complex thereof and/or one or more polynucleotide construct or complex thereof or modified polynucleotide construct or complex thereof encoding at least one therapeutic agent-containing composition when Tris buffer is present in a starting material can be diluted at least 1-fold, 2-fold, 5-fold, 10-fold, 100-fold or more with the pre-spray-drying liquid formulation not containing Tris buffer.

In some embodiments, the liquid formulation containing one or more polynucleotide construct or complex thereof or modified polynucleotide construct or complex thereof further includes, but is not limited to, one or more nonionic starch, or derivative thereof, other starch, polysaccharide, or substitutable agent thereof. In accordance with these embodiments, the nonionic starch or nonionic starch derivative, or substitutable agent thereof includes, but is not limited to, one or more of hydroxyethyl starch (HES), succinylated gelatin, and the like or combinations thereof. In some embodiments, at least one nonionic starch or derivative thereof, other starch, polysaccharide, or substitutable agent thereof, can be present in the aqueous or liquid formulation at a weight-to-volume (w/v) concentration from about 0.1% to about 40.0%, from about 1.0% to about 30.0%, from about 5.0% to about 20.0%, or from about 8.0% to about 15.0% or about 12.0%. In certain embodiments, the non-reducing disaccharide agent can be sucrose and/or trehalose and the at least one nonionic starch can be hydroxyethyl starch (HES).

In some embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent includes, but is not limited to, one or more one or more polymers. In accordance with these embodiments, the one or more polymers include, but are not limited to, polyvinyl alcohol (PVA), polyacrylic acid (PAA), polyethylene glycol (PEG), povidone, polymethacrylate-based copolymers (e.g., Eudragit), other medium to high molecular weight polymers or combinations thereof. It is contemplated herein that polymers with lower solubility can be preferred to facilitate the drying process. In some embodiments, compositions disclosed herein exclude polymers having a low molecular weight and/or have high solubility. In certain embodiments compositions disclosed herein having one or more polymers include, PVA, in part due to its lower solubility compared to other polymers. In some embodiments, compositions disclosed herein can include, but are not limited to, a liquid formulation including a single lipid nanoparticle or a mixture of lipid nanoparticles (LNPs) and further including at least one polymer for encapsulating and stabilizing the polynucleotide construct or complex thereof or modified polynucleotide construct or complex thereof, or a polynucleotide construct or complex thereof or modified polynucleotide construct or complex thereof encoding one or more therapeutic agents (e.g., polypeptide or chimera) thereof. In certain embodiments, these liquid formulations can then be spray-dried to an essentially dry powder or microparticles; and optionally, coated with one or more metalloorganic, metal oxide and/or metal alkoxide agent using ALD.

In some embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent further includes, but is not limited to, at least one cellulose agent. In accordance with these embodiments, the at least one cellulose agent comprises at least one of carboxymethyl cellulose (CMC) (low, medium, and/or high molecular weight), chitosan, pectin, dextran, or other cellulose agent, or a combination thereof.

In some embodiments, the liquid formulation (prior to drying) containing one or more polynucleotide construct or complex thereof or modified polynucleotide construct or complex thereof and/or one or more polynucleotide construct or complex thereof or modified polynucleotide construct or complex thereof encoding a therapeutic agent can include, but is not limited to, the one or more volatile salts. In accordance with these embodiments, the one or more volatile salts include, but are not limited to, one or more of: ammonium acetate, ammonium formate, ammonium carbamate, ammonium carbonate, ammonium bicarbonate, triethylammonium acetate, triethylammonium formate, triethylammonium carbonate, trimethylamine acetate trimethylamine formate, trimethylamine carbonate, pyridinal acetate and pyridinal formate, or combinations thereof. In some embodiments, the volatile salt is ammonium acetate. In certain embodiments, the liquid formulation (prior to drying) containing one or more polynucleotide construct or complex thereof or modified polynucleotide construct or complex thereof and/or one or more polynucleotide construct or complex thereof or modified polynucleotide construct or complex thereof encoding a therapeutic agent can specifically exclude a volatile salt and/or a volatile buffer.

In some embodiments, the liquid formulation containing one or more polynucleotide construct or complex thereof or modified polynucleotide construct or complex encoding at least one therapeutic agent thereof includes, but is not limited to, any form of one or more of DNA, siRNA, RNA (e.g., mRNA), other polynucleotide, or a mixture or complex thereof. In certain embodiments, the one or more polynucleotide construct or complex thereof or modified polynucleotide construct or complex thereof includes, but is not limited to, a polynucleotide or complex thereof encoding one or therapeutic agents comprises one or more antigens or immunogenic agent; or includes at least one polynucleotide capable of inducing an immune response in a subject or having specific and/or general immunomodulatory properties. In accordance with these embodiments, the one or more polynucleotide construct or complex thereof or modified polynucleotide construct or complex thereof and/or the one or more polynucleotide construct or complex thereof or modified polynucleotide construct or complex thereof encoding at least one therapeutic agent encodes at least one therapeutic agent capable of inducing an immune response in a subject or has immunomodulatory properties. In certain embodiments, polynucleotides disclosed herein are mRNAs capable of inducing an immune or other therapeutic response to reduce onset of, prevent and/or treat a health condition (e.g., cancer, an infection by a pathogen).

In some embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent thereof encodes at least one antigen specific to reduce onset of, or prevent infection in a subject by one or more of a pathogenic virus, a pathogenic bacteria, a fungal pathogen; a chimera, a toxin, or a tumor antigen or a combination thereof. In certain embodiments, the one or more polynucleotide construct or complex thereof or modified polynucleotide construct or complex thereof comprises at least one polynucleotide encoding a recombinant polypeptide or protein; a virus-like particle; a live virus; a live, an attenuated virus; an inactivated virus; a toxoid; or fragment thereof or a combination thereof.

In some embodiments, the liquid formulation containing the one or more polynucleotide construct or complex thereof or modified polynucleotide construct or complex thereof and/or the one or more polynucleotide construct or complex thereof or modified polynucleotide construct or complex thereof encoding at least one therapeutic agent encodes at least one antigen specific to or derived from, one or more of: human papilloma virus (HPV or type or serotype thereof), Ebola virus, poliovirus, norovirus, rotavirus, hepatitis A, hepatitis, B, hepatitis C, dengue virus, Lentivirus, human immunodeficiency virus (HIV), varicella-zoster virus, herpes simplex virus, cytomegalovirus, Japanese encephalitis virus, West Nile virus, Zika virus, Haemophilus influenzae type b, measles virus, mumps virus, rubella virus, respiratory syncytial virus, influenza virus, yellow fever virus, rabies virus, smallpox virus, parvovirus, chikungunya virus, other flaviviruses, other alphaviruses, Corona viruses (e.g., causes COVID-19, or derivative thereof) Corynebacterium diptheriae, Clostridium tetani, Clostridium botulinum, Bordetella pertussis, Streptococcus pneumoniae, Neisseria meningitides, Salmonella spp., Bacillus anthracis, Yersinia spp., B. pseudomallei (Bp), or a combination thereof. In accordance with these embodiments, these polynucleotide formulations can be spray-dried and ALD-coated or remain uncoated and later reconstituted into a pharmaceutically acceptable formulation for introduction to a human subject to reduce onset of, prevent and/or treat an infection thereof.

In some embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent encodes at least one antigen specific to or derived from, one or more of: canine parvovirus, canine distemper virus, canine adenovirus, rabies virus, canine parainfluenza virus, canine influenza virus, canine corona virus, measles virus, Bordetella bronchiseptica, Leptospira spp., Borrelia burgdorferi, or a combination thereof. In accordance with these embodiments, these polynucleotide formulations can be spray-dried and ALD-coated or remain uncoated and later reconstituted into a pharmaceutically acceptable formulation for introduction to a canine to reduce onset of, prevent and/or treat an infection thereof.

In some embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent encodes at least one antigen specific to or derived from, one or more of: feline herpesvirus 1, feline calicivirus, feline panleukopenia virus, rabies virus, feline leukemia virus, feline immunodeficiency virus, virulent systemic feline calicivirus, Chlamydophila felis, Pasteurella haemolytica, Bordetella bronchiseptica, or a combination thereof. In accordance with these embodiments, these polynucleotide formulations can be spray-dried and ALD-coated or remain uncoated and later reconstituted into a pharmaceutically acceptable formulation for introduction to a feline to reduce onset of, prevent and/or treat an infection thereof.

In other embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent encodes at least one antigen specific to or derived from, one or more of: Eastern equine encephalomyelitis virus (EEEV), Western equine encephalomyelitis virus (WEEV), Venezuelan equine encephalomyelitis virus (VEEV), rabies virus, Chikungunya virus, chikungunya, West Nile virus, equine influenza virus, equine herpesvirus, Streptococcus equi equi, Clostridium tetani, Neorickettsia risticii, Clostridium tetani, or a combination thereof. In accordance with these embodiments, these polynucleotide formulations can be spray-dried and ALD-coated or remain uncoated and later reconstituted into a pharmaceutically acceptable formulation for introduction to an equine to reduce onset of, prevent and/or treat an infection thereof. In some embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent encodes at least one antigen specific to or derived from, one or more of: bovine herpesvirus, parainfluenza type 3 virus, bovine viral diarrhea virus, bovine respiratory syncytial virus, Clostridium chauvoei, Clostridium septicum, Clostridium novyi, Clostridium perfringens type C, Clostridium perfringens type D, Pasteurella haemolytica, Clostridium haemolyticum, Chikungunya virus, or a combination thereof. In accordance with these embodiments, these polynucleotide formulations can be spray-dried and ALD-coated or remain uncoated and later reconstituted into a pharmaceutically acceptable formulation for introduction to a bovine to reduce onset of, prevent and/or treat an infection thereof.

In other embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent encodes at least one antigen specific to or derived from, one or more of: Marek's disease virus, reovirus, avian encephalomyelitis virus, avian influenza virus, avipoxviruses, chicken anemia virus, Pasteurella multocida, Newcastle disease virus, Riemerella anatipestifer, duck herpesvirus 1, duck hepatitis virus, or a combination thereof. In accordance with these embodiments, these polynucleotide formulations can be spray-dried and ALD-coated or remain uncoated and later reconstituted into a pharmaceutically acceptable formulation for introduction to a bird to reduce onset of, prevent and/or treat an infection thereof.

In other embodiments, the liquid formulation containing one or more polynucleotide construct or complex thereof or modified polynucleotide construct or complex thereof and/or one or more polynucleotide construct or complex thereof or modified polynucleotide construct or complex thereof encoding at least one therapeutic agent encodes at least one antigen specific to or derived from, one or more of: Cryptococcus spp., Aspergillus spp., Blastomyces spp., Candida albicans, Paracoccidioides spp., Sporothrix spp., Histoplasma capsulatum, Pneumocystis jirovecii, Coccidioides immitis, or a combination thereof. In accordance with these embodiments, these polynucleotide formulations can be spray-dried and ALD-coated or remain uncoated and later reconstituted into a pharmaceutically acceptable formulation for introduction to a subject to reduce onset of, prevent and/or treat an infection thereof.

In other embodiments, the liquid formulation containing one or more polynucleotide construct or complex thereof or modified polynucleotide construct or complex and/or one or more polynucleotide construct or complex thereof or modified polynucleotide construct or complex encoding at least one therapeutic agent is exposed to a spray-drying process disclosed herein in order to create essentially dry microparticles or particulates. In accordance with these embodiments, the essentially dry composition can then be coated or encapsulated with at least one layer of metal-containing material or encapsulation. In certain embodiments, the essentially dry composition can be coated by an atomic layer deposition (ALD) coating process or chemical vapor deposition (CVD) coating process. In accordance with these embodiments, each layer of the one or more outer coating layers on essentially dry microparticles disclosed herein can include metalloorganic, metal oxide and/or metal alkoxide compositions applied by an ALD or CVD process or the like. In certain embodiments, each layer of the one or more outer coating layers on essentially dry microparticles disclosed herein can include aluminum oxide, an aluminum alkoxide (e.g., alucone), silicon dioxide (SiO2), titanium dioxide (TiO2), or silicon nitride (Si3N4), zinc conjugate (Zn (ZnO₂)), alone or in a suitable combination compositions. In accordance with these embodiments, each of the one or more coating layer(s) can be about 0.1 nm to about 20 nm in thickness. In certain embodiments, the essentially dry microparticles can include coating layers sufficient to delay release or provide a timed-release or precise timed-release of the at least one agent from an outer coating layer and/or the central or innermost agent-containing microparticles. In certain embodiments, the one or more coating layer(s) or encapsulating layers disclosed herein can serve as an adjuvant to enhance an immune response in a subject against one or more of the encoded antigens. In certain embodiments, the one or more coating layer(s) can contain a concentration capable of inducing an immune response to the coated microparticles. In some embodiments, one or more coating layers encapsulating microparticles disclosed herein can include a single layer, 2, 3, 4, 5, up to 10, up to 20, up to 30, up to 40, up to 50, up to 100, up to 150, up to 200, up to 250 or more or any number of coating layers in between. In certain embodiments, stabilized microparticles disclosed herein can be used in a pharmaceutically acceptable formulation without coating to reduce onset of, prevent and/or treat a health condition.

In other embodiments, the essentially dry composition, microparticles, or particulates; or partially or completely coated or encapsulated essentially dry composition microparticles, or particulates can be reconstituted for further analysis or use. In accordance with these embodiments, a reconstitution buffer can include a pharmaceutically acceptable buffer of use for delivering the coated or uncoated microparticles harboring the one or more polynucleotide construct or complex thereof or modified polynucleotide construct or complex and/or the one or more polynucleotide construct or complex thereof or modified polynucleotide construct or complex encoding at least one therapeutic agent to a subject to treat, reduce onset of, or prevent a health condition. In accordance with these embodiments, the pharmaceutically acceptable buffer can further include at least one pharmaceutically acceptable excipient.

In certain embodiments, essentially dry (e.g., post spray-dried) formulations containing at least one polynucleotide construct or complex thereof or modified polynucleotide construct or complex thereof and/or at least one polynucleotide construct or complex thereof or modified polynucleotide construct or complex thereof encoding at least one therapeutic agent can be encapsulated or coated with one or more coating layers to produce coated microparticles of polynucleotide construct or complex thereof or modified polynucleotide construct or complex thereof. In certain embodiments, the coated or encapsulated microparticles can be coated for pre-determined timed-release delivery of the polynucleotide construct or complex thereof or modified polynucleotide construct or complex thereof and/or at least one polynucleotide construct or complex thereof or modified polynucleotide construct or complex thereof released encoding at least one therapeutic agent. In some embodiments, primary and boost doses of the coated microparticles housing the polynucleotide construct or complex thereof or modified polynucleotide construct or complex thereof can be administered to a subject in a single administration in the same or different coated microparticles. In other embodiments, coated polynucleotide construct or complex thereof or modified polynucleotide construct or complex thereof-containing microparticles can include immunogenic agents against one or more pathogens either in the same or in separate microparticles. In accordance with these embodiments, one, two, three, four, five or more coating layers as provided herein can encase the microparticles where the coating layers dissolve in a subject once administered or at a predetermined period depending on the number of layers applied and physiological conditions of the subject, and expose the polynucleotide construct or complex thereof or modified polynucleotide construct or complex thereof from within the now uncoated microparticles to the subject.

In other embodiments, the polynucleotide construct or complex thereof or modified polynucleotide construct or complex thereof-containing microparticles can increase stability and/or compatibility of a variety of typically incompatible polynucleotide construct or complex thereof or modified polynucleotide construct or complex thereof. In certain embodiments, two or more different polynucleotides can be sequestered in a core, and/or outer layering of coated microparticles where these two or more different polynucleotides without using stabilization and thermostability preparation and coating as disclosed herein would be incompatible and unable to deliver in a single administration to a subject in need thereof.

In other embodiments, pharmaceutical composition are contemplated of use herein that include a reconstituted composition of an essentially dried thermostable formulation containing at least one polynucleotide construct or complex thereof, and a pharmaceutically acceptable excipient. In certain embodiments, a pharmaceutical composition contemplated herein can be used as a vaccine of use against one or more pathogens, or against cancer, or in delivery of a polynucleotide-containing formulation for gene therapy. In accordance with these embodiments, gene therapy can include protein replacement therapy. In some embodiments, gene therapies contemplated herein can include gene replacement therapy to treat, ameliorate, or cure a health condition, including, but not limited to, cystic fibrosis, phenylketonuria, hemophilia, Type 2 diabetes, myocardial ischemia, myocardial ischemia, liver fibrosis, lung fibrosis, COPD, or other health condition or combination thereof. It is contemplated herein that any health condition treatable by protein replacement in a gene therapy setting can be treated by compositions and methods disclosed herein for delivery of at least one targeted protein or polynucleotide encoding at least one targeted protein to treat the health condition in a subject.

Other embodiments include methods for preparing a composition disclosed herein, In certain embodiments, method can include combining the components of a formulation disclosed herein including at least one polynucleotide construct or complex thereof or modified polynucleotide construct or complex thereof, or at least one polynucleotide construct or complex thereof or modified polynucleotide construct or complex thereof encoding one or more therapeutic agents, and one or more non-reducing disaccharide and one or more polymer to create a liquid formulation; and at least one of: atomizing the liquid formulation in a gas stream to create particulates and spray-drying the liquid formulation to create an essentially dry formulation. In some embodiments, at least 1.0% of the polynucleotide construct or complex thereof or modified polynucleotide construct or complex thereof encoding one or more therapeutic agents is stable or wherein less than 99.0%, or less than 80%, or less than 70.0% or less than 60% or less than 50% or less than 40% of the polynucleotide construct or complex thereof or modified polynucleotide construct or complex thereof encoding one or more therapeutic agents is degraded.

In certain embodiments, a composition disclosed herein can be stable for at least one month, or more at elevated temperatures; optionally, where elevated temperatures are at least room temperature up to about 70° C. In other embodiments, the one or more non-reducing disaccharide is present in a weight-to-volume concentration from about 1.0% to about 40.0% in the liquid formulation. In some embodiments, the at least one non-reducing disaccharide can include at least one of trehalose, sucrose, lactose, other non-reducing disaccharide, or the like or a combination thereof. In certain embodiments, the at least one glass-forming agent can include trehalose. In accordance with this embodiment, trehalose can be included as a non-reducing disaccharide or glass-forming agent and can be present in the primary liquid immunogenic composition in a weight-to-volume (w/v) concentration of from about 0.1% to about 40.0%, from about 1% to about 30.0%, from about 5.0% to about 20.0%, or from about 8.0% to about 15.0%, or about 12.0%.

In certain embodiments, when spray-drying a liquid formulation disclosed herein, several gases can be used for these processes. In accordance with these embodiments, these gases can be referred to as process gases. In certain embodiments, process gases can include, but are not limited to, ambient air, dehumidified air, nitrogen, or inert gas as one or all of nozzle, cyclone, or drying gas to spray-dry formulations disclosed herein to form particulates or essentially dry microparticles.

In certain embodiments, during the spray-drying process, gas used for the spray-drying process can have an inlet gas or nozzle temperature of less than 200° C., less than 190° C., or less than 180° C., or less than 170° C., or less than 160° C., or less than 150° C., or less than 140° C. or less than 130° C., or less than 120° C., or less than 110° C., or less than 100° C. or less than 90° C., or less than 80° C. or less than 70° C., or less than 60° C., or 50° C. In certain embodiments, when a volatile buffer or an additional alcohol is present in an aqueous or liquid formulation, an inlet gas or nozzle temperature can be from about 25° C. to about 200° C., or about 25° C. to about 120° C., or about 25° C. to about 100° C., or about 25° C. to about 80° C. or about 25° C. to about 65° C., or about 25° C. to about 55° C., enhancing the drying process and reducing the need for elevated nozzle or inlet gas temperatures. In some embodiments, drying gas used for the spray-drying process has a relative humidity less than about 50%, or less than about 40%, or less than about 30%, or less than about 20%, or less than about 10% or less than about 5%. In accordance with these embodiments, the aqueous or liquid formulation can further include at least one alcohol, and a lower drying temperature can be used for spray-drying the liquid formulation. In other embodiments, drying gas flow rates for the spray-drying process can be from about 0.01 to about 5.0 m3/min, or about 0.1 to about 2.5, or about 0.1 to about 1.5 m3/min, or about 0.1-1.2 m3/min. In certain embodiments, atomizing gas flow rates can be from about 0.01 to about 100.0 L/min or about 0.05 to about 75.0 L/min, or about 0.01 to about 75.0 L/min, or about 0.1 to about 50.0 L/min. In yet other embodiments, feed flow rates for the spray-drying process can be about 0.01 to about 50.0 g/min, about 0.05 to about 40.0 g/min, or about 0.1-35 g/min. In certain embodiments, cyclone gas flow rates can be about 1.0 to about 1,000 L/min, about 1.0 to about 750.0 L/min, about 1.0 to about 500.0 L/min, or about 1.0 to about 400.0 L/min, or about 1.0 to about 300.0 L/min.

In certain embodiments, during the spray-drying process, a spray dryer for the spray-drying process can include a two-fluid atomizing nozzle, an ultrasonic atomizing nozzle, or any nozzle determined to effectively atomize the liquid formulation disclosed herein. In other embodiments, liquid formulations disclosed herein can further include at least one alcohol and the liquid formulation can be fed into a spray-dryer at a rate such that any drying and nozzle gas maintains the spray dryer below the lower flammability limit for any evolved volatile components and reduces a chance of alcohol catching on fire.

In some embodiments, the at least one therapeutic agent can include one or more polynucleotide encoding at least one antigen. In accordance with these embodiments, the at least one antigen can include, but is not limited to, at least one viral antigen, bacterial antigen, fungal antigen, protozoan-derived agent, toxin, fragment thereof, or a combination thereof. In some embodiments, at least one polynucleotide construct or complex thereof or modified polynucleotide construct or complex thereof can further include another agent including, but not limited to, at least one polypeptide, two or more different polynucleotides, a capsomere, one or more lipids, one or more lipid nanoparticles (LNPs), or other polynucleotide complexable agents or the like.

In certain embodiments, methods for eliciting an immune response against one or more pathogenic organisms or stimulate an immune response against cancer or other antigens in a subject are provided. In accordance with these embodiments, a pharmaceutical composition disclosed herein can be administered to a subject, where the polynucleotide construct or complex thereof or modified polynucleotide construct or complex thereof and/or the polynucleotide construct or complex thereof or modified polynucleotide construct or complex thereof encoding one or more therapeutic agents encodes one or more immunogenic agent derived from at least one pathogenic organism, against cancer, or other antigen capable of inducing a response to treat a health condition, and/or elicits an immune response in the subject to the one or more polynucleotide construct or complex thereof or modified polynucleotide construct or complex thereof to reduce onset of, prevent or treat an infection, cancer or the like.

In some embodiments, stabilized microparticles (e.g., essentially dry) and/or coated or encapsulated microparticles disclosed herein can be stored without refrigeration temperatures of up to about 40° C. to about 70° C. for extended periods of time. In certain embodiments. stabilized microparticles (e.g., essentially dry) and/or coated or encapsulated microparticles disclosed herein can be stored without refrigeration up to about 40° C. to about 70° C. up to about 1 month, up to about 2 months, up to about 3 months, or up to about 4 months, or up to about 6 month, or up to about 9 months, or up to about 12 months, or up to about 15 months, or up to about 18 months, or up to about 24 months or longer without negative effect on the polynucleotide construct or complex thereof or modified polynucleotide construct or complex thereof and/or the polynucleotide construct or complex thereof or modified polynucleotide construct or complex thereof encoding one or more therapeutic agents (e.g. degradation). Therefore, these processes can be used to more accurately deliver a given antigen or agent for reduced costs in production, storage, delivery, and administration (e.g., more than one dose in a singly administered composition)

In some embodiments, stabilized microparticles (e.g., essentially dry) and/or coated or encapsulated microparticles disclosed herein can be reconstituted into a single-administration composition of a prime dose and boost dose of the at least one encoded therapeutic agent. In accordance with these embodiments, the prime and boost doses of the at least one encoded therapeutic agent can be in the same coated or encapsulated microparticle, or in separate microparticles. When in separate microparticles, the priming dose of the at least one encoded therapeutic agent can be sequestered in an essentially dry spray-dried state without any outer coating layers, or in a coated microparticle(s), while a boost dose of the at least one encoded therapeutic agent is in another microparticle. Whether the priming dose is in a coated microparticle or uncoated microparticle will be determined by whether an immediate or delayed response is desired, where outer coating layers sequestering the prime dose will temporally delay release of the priming dose, and therefore, delay exposure of the priming dose to the subject.

In some embodiments, stabilized microparticles (e.g., essentially dry) and/or coated or encapsulated microparticles disclosed herein can be a single administration composition capable of eliciting an immune response to two or more different encoded therapeutic agents. In accordance with these embodiments, the two or more different encoded therapeutic agents can be included in the same coated or uncoated microparticles, or in separate coated or uncoated microparticles. In other embodiments, when the two or more different encoded therapeutic agents are contained in separate microparticles, each different encoded therapeutic-containing particle can include a different encoded or translated therapeutic agent as desired.

In some embodiments, pharmaceutical compositions disclosed herein can include a standard vaccine composition and a plurality of uncoated or coated microparticles described. In yet other embodiments, pharmaceutical compositions disclosed herein can include a plurality of a first polynucleotide construct or complex thereof or modified polynucleotide construct or complex thereof and/or the polynucleotide construct or complex thereof or modified polynucleotide construct or complex thereof encoding one or more therapeutic agents containing coated or uncoated microparticles described herein, and optionally, at least a second polynucleotide construct or complex thereof or modified polynucleotide construct or complex thereof and/or the polynucleotide construct or complex thereof or modified polynucleotide construct or complex thereof encoding one or more therapeutic agents containing coated or uncoated microparticles described herein. In accordance with these embodiments, the plurality of second polynucleotide construct or complex thereof or modified polynucleotide construct or complex thereof and/or the polynucleotide construct or complex thereof or modified polynucleotide construct or complex thereof encoding one or more therapeutic agents containing coated or uncoated microparticles described herein is the same or different than the first polynucleotide construct or complex thereof or modified polynucleotide construct or complex thereof and/or the polynucleotide construct or complex thereof or modified polynucleotide construct or complex thereof encoding one or more therapeutic agents containing coated or uncoated microparticles described herein; and further includes a pharmaceutically acceptable excipient. In accordance with embodiments, these compositions can further include a plurality of at least one additional microparticle described herein, wherein the at least one additional microparticle includes at least one additional agent that is not the first agent or the second agent.

Other embodiments provide for methods for eliciting an immune response in a subject, where the method can include administering a composition described herein to the subject. In accordance with these embodiments, the composition can induce an immune response in the subject. In accordance with these embodiments, the immune response induced by the composition can be prophylactic and/or therapeutic depending on the immunogenic agent.

In certain embodiments, formulations disclosed herein including at least one polynucleotide construct or complex thereof or modified polynucleotide construct or complex thereof and/or the polynucleotide construct or complex thereof or modified polynucleotide construct or complex thereof encoding one or more therapeutic agents containing coated or uncoated microparticles disclosed herein can use less agent than used to formulate current therapies (e.g., cost saving), and provide enhanced efficacy after a single administration. In other embodiments, a coated or uncoated microparticle-containing formulation disclosed herein eliminate and/or reduce refrigeration requirements (e.g., cold chain refrigeration requirements), limit the concentration of adverse agents (e.g., aluminum) administered to subjects, and increase agent compatibility. In other embodiments, compositions and methods disclosed herein are applicable to a variety of therapeutic agents, including, but not limited to, any therapeutic polynucleotide, any encoded antigen or genetic material for reducing onset, preventing, ameliorating, or treating a health condition.

In certain embodiments, a polynucleotide or an encoded therapeutic agent can be directed to any pathogenic virus, for example, a papovavirus (e.g., papillomaviruses, including human papilloma virus (HPV)), a herpesvirus (e.g., herpes simplex virus, varicella-zoster virus, bovine herpesvirus-1, cytomegalovirus), a poxvirus (e.g., smallpox virus), a reovirus (e.g., rotavirus), a parvovirus (e.g., parvovirus B19, canine parvovirus), a picornavirus (e.g., poliovirus, hepatitis A), a togavirus (e.g., rubella virus, alphaviruses such as chikungunya virus), a hepadnavirus (e.g., hepatitis B virus), a flavivirus (e.g., dengue virus, hepatitis C virus, West Nile virus, yellow fever virus, Zika virus, Japanese encephalitis virus), an orthomyxovirus (e.g., influenza A virus, influenza B virus, influenza C virus), a paramyxovirus (e.g., measles virus, mumps virus, respiratory syncytial virus, canine distemper virus, parainfluenza viruses), a rhabdovirus (e.g., rabies virus), a filovirus (e.g., Ebola virus), or a coronavirus or combinations thereof.

In other embodiments, a polynucleotide or an encoded therapeutic agent can be directed to bacterium or a toxin of a bacterium, including but not limited to, Pasteurella haemolytica, Clostridium difficile, Clostridium haemolyticum, Clostridium tetani, Corynebacterium diphtheria, Neorickettsia resticii, Streptococcus equi equi, Streptococcus pneumoniae, Salmonella spp., Chlamydia trachomatis, Bacillus anthracis, Yersinia spp., and Clostridium botulinum or combinations thereof.

In some embodiments, a polynucleotide or an encoded therapeutic agent can be directed to a fungus, including but not limited to Cryptococcus spp. (e.g., neoformans and gatti), Aspergillus spp. (e.g., fumigatus), Blastomyces spp. (e.g., dermatitidis), Candida albicans, Paracoccidioides spp. (e.g., brasiliensis), Sporothrix spp. (e.g., schenkii and brasiliensis), Histoplasma capsulatum, Pneumocystis jirovecii and Coccidioides immitis, or combinations thereof.

In yet other embodiments, a polynucleotide or an encoded therapeutic agent can be directed to a toxin, such as ricin toxin or botulinum toxin. In some embodiments, formulations described herein can be used to manufacture one or more composition of use as vaccines for animals such as household pets. In accordance with these embodiments, the immunogenic composition can be administered, for example, to a dog (canine), a cat (feline), a horse (equine), cattle (bovine), a goat (hircine), a sheep (caprine), or poultry (e.g., chicken, turkey, duck, goose).

In certain embodiments, a polynucleotide or an encoded therapeutic agent can be described herein can be used to generate one or more immunogenic compositions for administering to a canine to reduce onset of or prevent an infection, including but not limited to, infections related to canine parvovirus (CPV), canine distemper virus (CDV), canine adenovirus (CAV), rabies, canine parainfluenza virus (CPiV), canine influenza virus, canine corona virus, measles virus, Bordetella bronchiseptica, Leptospira spp., and Borrelia burgdorferi or combinations thereof.

In some embodiments, a polynucleotide or an encoded therapeutic agent can be described herein can be used to generate compositions of use for administering to a feline to reduce or prevent an infection or treat an infection, including but not limited to, compositions directed to feline herpesvirus 1 (FHV1), feline calicivirus (FCV), feline panleukopenia virus (FPV), rabies, feline leukemia virus (FeLV), feline immunodeficiency virus, virulent systemic feline calicivirus, Chlamydophila felis, Pasteurella haemolytica, and Bordetella bronchiseptica or combinations thereof.

In other embodiments, a polynucleotide or an encoded therapeutic agent described herein can be used to generate compositions of use for administering to equine, to reduce or prevent an infection or treat an infection, including but not limited to, compositions directed to Eastern equine encephalomyelitis virus, Western equine encephalomyelitis virus, Venezuelan equine encephalomyelitis virus, bovine papillomavirus, rabies virus, Clostridium tetani, West Nile virus, equine influenza virus, Potomac fever (Neorickettsia risticii), Streptococcus equi equi, and rhinopneumonitis (equine herpesevirus type 1) or combinations thereof.

In certain embodiments, a polynucleotide or an encoded therapeutic agent described herein can be used to generate compositions of use for administering to bovine, to reduce or prevent an infection or treat an infection, including but not limited to, compositions directed to bovine rhinotracheitis (IBR), parainfluenza type 3 (PI3), bovine virus diarrhea (BVD), bovine respiratory syncytial virus (BRSV), blackleg (Clostridium chauvoei), malignant edema (Clostridium septicum), infectious necrotic hepatitis (Clostridium novyi), enterotoxemia (Clostridium perfringens type C and D), Pasteurella haemolytica, and redwater (Clostridium haemolyticum) or combinations thereof.

In some embodiments, a polynucleotide or an encoded therapeutic agent described herein can be used to generate compositions of use for administering to poultry, to reduce or prevent an infection or treat an infection, including but not limited to, immunogenic compositions directed to Marek's disease (Marek's disease virus), tenosynovitis (reoviruses), encephalomyelitis (avian encephalomyelitis virus), fowlpox (avipoxviruses), chicken infectious anemia (chicken anemia virus), fowl cholera (Pasteurella multocida), Newcastle/infectious bronchitis (Newcastle disease virus), Riemerella anatipestifer, duck viral hepatitis (duck hepatitis virus), and duck viral enteritis (duck herpesvirus 1) or combinations thereof.

In some embodiments, a polynucleotide or an encoded therapeutic agent described herein can be used to generate compositions of use for administering to a human. In certain embodiments, a polynucleotide and/or an encoded therapeutic agent described herein can be used to deliver one or more immunogenic compositions to a human such as a fetus, an infant, a toddler or child or adolescent, young adult, adult or elderly adult, including but not limited to vaccines for varicella-zoster (chicken pox), diphtheria, Haemophilus influenzae type b (Hib), HPV (all pathogenic types), hepatitis A, hepatitis B, influenza, measles, mumps, pertussis, polio, pneumococcal disease, rotavirus, rubella, and tetanus. In other embodiments, immunogenic agent-containing particles described herein may be used to deliver one or more immunogenic compositions to a human pre-teen or teen, including but not limited to vaccines for influenza, tetanus, diphtheria, pertussis, human papillomavirus, meningococcal disease, hepatitis B, hepatitis A, polio, measles, mumps, rubella, and varicella-zoster. In yet other embodiments, immunogenic agent-containing particles described herein may be used to deliver one or more immunogenic compositions to a human adult, including but not limited to immunogenic compositions against influenza (e.g., A, B or C), tetanus, diphtheria, pertussis, zoster, pneumococcal disease, meningococcal disease, measles, mumps, rubella, varicella, hepatitis A, hepatitis B, and Haemophilus influenzae type b.

In other embodiments, a polynucleotide or an encoded therapeutic agent described herein can be used to generate immunogenic compositions of use for administering to a human, including but not limited to, immunogenic compositions against travel-related diseases or any pathogenic organism thereof, including but not limited to, reducing onset of, preventing and/or treating, hepatitis A, hepatitis B, paratyphoid fever, meningococcal disease, yellow fever, Zika infection, dengue fever, rabies, Chikungunya disease, typhoid fever, malaria, covid or covid variant, and Japanese encephalitis or other flavivirus or alphavirus-related condition.

In other embodiments, a polynucleotide or an encoded therapeutic agent described herein can be used to generate immunogenic compositions of use for administering to a human, including but not limited to, immunogenic compositions against human papillomavirus (e.g. HPV 16, HPV18, HPV31, HPV45, or HPV 6 or HPV11, or any other HPV), HIV, lentivirus, coronavirus (e.g., CoV2, COVID-19, Middle East Respiratory Syndrome (MERS-CoV) and Severe Acute Respiratory Syndrome (SARS-CoV), novel coronavirus (nCoV) etc), herpes simplex virus, smallpox virus, rotavirus, parvovirus B19 vaccine, chikungunya virus, dengue virus (e.g. dengue-1, dengue-2, dengue-3 or dengue-4), hepatitis C virus, West Nile virus, respiratory syncytial virus, and the like or combinations thereof.

In some embodiments, one or more agents supplementing formulation pre-or post-spray-drying, such as an adjuvant, can be included; for example, in an essentially dry microparticle after the spray-drying process. In accordance with these embodiments, supplementing agents contemplated herein can include, but are not limited to, one or more one aluminum-salt adjuvants, one or more buffers containing one or more one volatile salts, one or more immunologically-related agents, one or more smoothing excipients, and co-stimulatory agents (e.g., co-immunostimulatory agents).

In other embodiments, buffers of use for reconstitution of ALD-coated or uncoated essentially dry microparticles disclosed herein can include, but are not limited to, acetate, succinate, citrate, prolamine, histidine, borate, carbonate, or phosphate buffer, water, saline, or a combination thereof. In accordance with these embodiments, water can be used for reconstitution and administration of a reconstituted composition disclosed herein. In certain embodiments, a buffer can include one or more preservatives, if needed. In some embodiments, the buffer can include histidine or borate buffer. In certain embodiments, the buffer can include histidine, for example, histidine-HCl. In certain embodiments, the buffer can include, but is not limited to, HES alone or in combination.

In certain embodiments, compositions disclosed herein can include a co-stimulatory agent in order to boost immune responses to an immunogenic agent against a pathogen. In accordance with these embodiments, a co-immunostimulatory agent can include, but is not limited to, one or more of lipid A, lipid A derivatives, monophosphoryl lipid A, chemical analogues of monophosphoryl Lipid A, CpG containing oligonucleotides, TLR-4 agonists, flagellin, flagellins derived from gram negative bacteria, TLR-5 agonists, fragments of flagellins capable of binding to TLR-5 receptors, saponins, analogues of saponins, QS-21, purified saponin fractions, ISCOMS and saponin combinations with sterols and lipids, or combinations thereof. In certain embodiments, the one or more coating layers coating microparticles disclosed herein eliminate the need for an adjuvant and further eliminate the need for a co-stimulatory agent. In accordance with these embodiments, liquid formulations disclosed herein to generate coated microparticles and/or reconstituted coated microparticles for use in a subject can specifically exclude an unrelated adjuvant agents and/or costimulatory agents, reducing occurrence of adverse effects due to the presence of these additional agents.

In certain embodiments, it is understood in the art that as complexity of a vaccine or immunogenic composition increases, long term stability typically decreases, for example, when temperatures are elevated such as during storage or transport vaccine complexes or constructs degrade more rapidly. In certain embodiments, immunogenic compositions having multiple subunits (e.g., multimeric) can be more complex than compositions of single encoded agents. For example, immunogenic compositions having antigens based on multiple capsomere subunit types can be less stable immunogenic compositions than a single subunit type. Compositions and methods disclosed herein create more stable complexes when in elevated temperatures during transport, storage and delivery.

In some embodiments, immunogenic agents encoded by polynucleotides disclosed herein can be of use for prophylactic and/or therapeutic treatment of a health condition. Suitability of immunogenic agents for use in immunogenic agent-containing particles can be tested by reaction with antibodies or monoclonal antibodies which react or recognize conformational epitopes present on the intact target of the immunogenic agent and based on the immunogenic agent's ability to elicit the production of neutralizing antiserum. Suitable assays for determining whether neutralizing antibodies are produced are known to those of skill in the art. In this manner, in certain embodiments, it can be verified whether the immunogenic agents of the present disclosure will elicit production of neutralizing antibodies.

In some embodiments, molecular deposition techniques can be used to apply nanometer-thick coatings of inorganic, organic, or metallo-organic materials on the surface of essentially dry microparticles disclosed herein. In certain embodiments, the one or more coating layers can be a metal oxide and/or metal alkoxide material. In yet other embodiments, the one or more coating layers can be an aluminum-based material including, but not limited to, an aluminum oxide or an aluminum alkoxide (e.g., alucone). In accordance with these embodiments, the metal ion and/or aluminum-containing material is deposited on or applied to the surface of the one or more particulates or essentially dry microparticles b to coat or sequester the one or more immunogenic agent-containing glassy microparticles in 1, 2, 3, 4, 5, 6, 7, 8, 9, 10, 11 or more layers of the aluminum-containing material to form encased immunogenic agent-containing glassy microparticles.

In certain embodiments, coating layers other than aluminum-based coating layers can be used in order to coat or sequester the immunogenic agent containing glassy microparticles. In accordance with these embodiments, non-aluminum coating layers can include, but are not limited to, silicon dioxide (SiO₂), titanium dioxide (TiO₂), or silicon nitride (Si₃N₄) or zinc which can be used either in combination with aluminum-based coating layers, or alone to the exclusion of aluminum-based coating layers or in an alternating pattern if suitable or desired. With each type of material having different characteristic dissolution times, layers of different materials can be deposited on the microparticles to vary the temporal release of the at least one therapeutic agent from the microparticle's core. In some embodiments, one or more aluminum-based layers can be introduced, followed by one or more layers of a different material. In accordance with these embodiments, different materials may dissolve more slowly than the aluminum-based coating layer. Using other materials for coating the particles, can reduce the number of aluminum-based layers necessary to provide for a given release time, minimizing the amount of aluminum per dose.

In other embodiments, one or more coating layers can be deposited on the particulates or essentially dry microparticles by, for example, atomic layer deposition (ALD, for example any instrumentation capable of atomic layer deposition can be used). ALD includes a thin film deposition technique that is based on the sequential use of a gas phase chemical process within an ALD reaction chamber. ALD is considered a type of chemical vapor deposition. In certain methods, most ALD reactions use two chemicals, referred to as precursors. These precursors react with the surface of a material one at a time in a sequential, self-limiting, or directed manner providing precision and complete coating of the particulates and/or essentially dry microparticles disclosed herein. Through the repeated exposure to separate precursors, a thin film can be slowly deposited. Use of ALD to deposit coating layers on the microparticles can be based on sequential, self-limiting reactions and provides for layer thickness control at the Angstrom level and tunable coating layer composition as previously disclosed. In accordance with these embodiments, particulates and/or essentially dry microparticles disclosed herein are formulated and spray dried such that polynucleotides such as high molecular weight polynucleotides are not principally located at the surface of the spray dry particle (reduced exposure). It is known that high molecular weight compounds and low solubility compounds have a higher likelihood of ending up at the surface of a spray dried particle, therefore, embodiments disclosed herein address this commonly observed issue by specific additions to the formulation to form an external shell-like layer that is not principally polynucleotides and can, for example, shield the polynucleotides making them more stable under certain conditions. In accordance with these embodiments, particulates and/or essentially dry microparticles disclosed herein can be introduced to an ALD reaction chamber where the particulates and/or essentially dry microparticles flow freely within the chamber for reduced agglomeration and/or aggregation of the particulates and/or essentially dry microparticles. It is noted herein that introduction of polynucleotides and/or polynucleotides encoding at least one therapeutic agent directly to an ALD reaction chamber without creating thermostable particulates or essentially dry glassy microparticles as disclosed herein would be an unsuccessful coating protocol; for example, the elevated temperature would degrade the polynucleotides and/or polynucleotides encoding at least one therapeutic agent; the polynucleotides and/or polynucleotides encoding at least one therapeutic agent would likely stick to one another or the chamber and/or the metal material applied would not layer onto free polynucleotides and/or fully encapsulate the polynucleotide and/or the polynucleotide encoding the at least one therapeutic agent because there would be no availability of binding groups needed to secure the coating which is created herein using polymer-containing formulations to create the essentially dry microparticles or particulates disclosed herein for long term storage or for further coating. It was not known until the instant disclosure that formulations disclosed herein could create such a barrier or shell to protect polynucleotides from such conditions. It is understood by one of skill in the art that formulations and processes disclosed herein are scalable and readily available for manufacture for creating bulk microparticles for coating or storage and later coating.

In certain embodiments, the ALD coating method can be optimized for a particular agent, situation or condition. For example, polynucleotides and/or polynucleotides encoding a therapeutic agent against a pathogenic organism of essentially dry microparticles disclosed herein can have variable thermostability, and therefore might not be amenable to higher ALD temperatures due to this vulnerability. In certain embodiments, molecular deposition can occur at temperatures at which the at least one polynucleotides and/or polynucleotides encoding a therapeutic agent of the essentially dry microparticles remains stable. In some embodiments, molecular deposition can occur under vacuum conditions. By performing the molecular deposition under vacuum conditions, coating layers can be applied at reduced temperatures thereby reducing adverse effects of higher temperatures on polynucleotides and/or polynucleotides encoding a therapeutic agent sequestered within the essentially dry microparticles. In certain embodiments, vacuum conditions required for deposition can be minimal. In some embodiments, the ALD process can occur under a mild vacuum of about 0.001-0.1 atmospheres. In other embodiments, ALD can also include incorporation of mechanical powder agitation devices that can lead to shorter cycle times for deposition of the material by providing uniform distribution of powders and reactants within an ALD reactor, avoiding clumping and agglomeration of the microparticles as well as ensuring complete coating layers with reduced or eliminated pinholes, for example.

Certain embodiments include methods to elicit an immune response in a subject to a polynucleotide or polynucleotide encoding at least one therapeutic agent encased in a particulate or essentially dry microparticle or combination of microparticles disclosed herein, by administering to the subject a composition including microparticles disclosed herein. The composition including the microparticles (e.g., coated, or uncoated) can be administered in therapeutically effective amounts. That is, in amounts sufficient to produce a protective immunological response, for example, against a pathogenic organism. Generally, the coated or uncoated particulate or microparticle-containing compositions disclosed herein can be administered in active agent dosages ranging from about 0.001 mg to about 100.0 mg. In certain embodiments, single or multiple dosages can be formulated within a single administration for providing to a subject. Where multiple doses are administered in a single administration composition, a prime and boost composition can be included, one of the two doses can be temporally controlled for release at a pre-selected time after administration.

In certain embodiments, administration of reconstituted compositions disclosed herein can be performed using any acceptable means (e.g., parenterally, locally, or systemically, including by way of example, oral, intranasal, intravenous, subcutaneous, intradermal, intravaginal, by suppository, intramuscular, intravaginal, intrauterine, and topical administration). In some embodiments, administration of reconstituted compositions disclosed herein can be by subcutaneous injection or intramuscular injection of a single composition to reduce onset of or prevent infection by one or more pathogenic organism. In some embodiments, administration can include direct administration to an affected site or directly into a tumor. In other embodiments, administration reconstituted compositions disclosed herein can be affected by factors including a natural route of infection by a particular pathogen. Dosage or dosages administered can depend upon factors including the age, health, weight, kind of concurrent treatment, exposure, if any, and the nature and type of the targeted agent.

### Kits

Other embodiments provide kits of use for storage and delivery of compositions disclosed herein. In addition, kits are disclosed for use in methods of making compositions disclosed herein. In certain embodiments, a kit can contain one more polynucleotide-containing microparticles in an essentially dry form either coated or uncoated. Different microparticles can be provided mixed together, or separate. In certain embodiments, different coated or uncoated microparticles are provided mixed together in a single container in a kit or found in separate containers for optional mixing. Kits can include one or more of an aqueous composition or essentially dry microparticles disclosed herein for coating by ALD or remaining uncoated for later use.

In other embodiments, kits are contemplated of use for compositions, and methods described herein. Kits can be portable. In certain embodiments, kits can be used to transport to and be used in remote areas such as military installations or remote villages. The thermostability of the immunogenic agent-containing glassy microparticles and immunogenic agent-containing particles allows for transport and storage without the need for a cold chain (e.g., refrigeration). This can also be advantageous in healthcare facilities, as it reduces costs associated with cold storage.

In other embodiments, kits can include an appropriate carrier or diluent suitable for reconstituting coated or uncoated particulates and/or essentially dry microparticles disclosed herein. In certain embodiments, the carrier or diluent can be a pharmaceutically acceptable aqueous buffer suitable for injection that includes pyrogen-free water and can resist changes in pH upon addition of an inorganic compound, organic compound, acid, alkali, or dilution with a solvent or diluent. In certain embodiments, kits can include the diluent and the coated or uncoated particulates and/or essentially dry microparticles in separate containers for ready reconstitution and use.

In some embodiments, kits can include one or more suitable containers, for example, vials, tubes, mini- or microfuge tubes, test tube, flask, bottle, syringe, or other container. Where an additional component or agent is provided, the kit can contain one or more additional containers into which this agent or component may be placed. Kits herein will also typically include a means for containing the immunogenic agent-containing agents and/or immunogenic agent-containing glassy microparticles, pharmaceutically acceptable carrier or diluent and any other reagent containers in close confinement for commercial sale. Such containers may include injection or blow-molded plastic containers into which the desired vials are retained.

In yet other embodiments, kits can include essentially dry microparticles of a polynucleotide or modified polynucleotide construct or complex thereof and/or a polynucleotide or modified polynucleotide construct or complex thereof encoding at least one therapeutic agent and compositions and instructions for coating the essentially dry microparticles of a polynucleotide or modified polynucleotide construct or complex thereof and/or a polynucleotide or modified polynucleotide construct or complex thereof encoding at least one therapeutic agent by ALD. In other embodiments, the kit can include an apparatus for performing such coatings on essentially dry microparticles of a polynucleotide or modified polynucleotide construct or complex thereof encoding at least one therapeutic agent. These coating layers provide for a level of temporally controlled release desirable with certain pharmaceutical agents. The coating layers can serve to reduce exposure to moisture, reducing degradation. These coatings can function to protect water-soluble formulations or other moisture sensitive agents from degradation or dissolution until desired exposure to a subject after administration.

### EXAMPLES

The materials, methods, and embodiments described herein are further defined in the following Examples. Certain embodiments are defined in the Examples herein. It should be understood that these Examples, while indicating certain embodiments, are given by way of illustration only. From the disclosure herein and these Examples, one skilled in the art can ascertain the essential characteristics of this invention, and without departing from the spirit and scope thereof, can make various changes and modifications of the invention to adapt it to various usages and conditions.

### Stabilization of Polynucleotide Compounds and Construct

In one exemplary method, powders with and without a polynucleotide construct are referred to herein as Lot 1 and Lot 2, respectively, and were manufactured with the intended use as initial test material for cell-based assays.

In certain exemplary methods, Lot 1 and Lot 2 were tested under previously disclosed standard formulation and spray dryer setpoints with LNP encapsulated mRNAs in Lot 2.

### Example 1

### Formulation 1

An aqueous solution of a formulation as found in Table I was made in 80% of a target volume. Components were added individually in the order listed and allowed to fully dissolve prior to adding the next. The pH of the solution was measured and adjusted to approximately pH 7.4 +/-0.2. The aqueous solution was sterilized through a 0.22 um filter. The mRNA-LNP construct (e.g., GreenLantern mRNA) was spiked into the aqueous solution, or was omitted to prepare a placebo. The pH was again measured and adjusted to pH 7.4 +/-0.2 where needed. Endotoxin-free water was added to target volume. In this example, the liquid formulation was stored on ice for approximately 15 minutes until the time of spray-drying. LNPs do not settle in this formulation. No phase separation was observed (e.g., for 12 hours).

TABLE 1
Composition of Formulation 1 as a liquid and spray dried product

| Component | Liquid w/w | Spray dried w/w |
|---|---|---|
| Trehalose | 0.106 | 0.640 |
| Hydroxyethyl starch | 0.028 | 0.171 |
| Ammonium acetate | 0.003 | Trace ~0.00003 |
| Histidine | 0.001 | 0.005 |
| Formulated mRNA-LNP | 0.027 | 0.164 |
| Ultrapure water | Quantity Sufficient to | — |
| | target volume (QS) | |

Additional Formulation Examples:

### Example 2

### Formulation 2

In another example, the following formulation was tested for effects on polynucleotide complexes for use in spray-drying techniques. In this example, aqueous solution of formulations illustrated in Table 2 were used and made according to the details in Example 1 (above).

TABLE 2
Composition of Formulation 2 as a liquid and spray dried product

| Component | Liquid w/w | Spray dried w/w |
|---|---|---|
| Sucrose | 0.106 | 0.640 |
| Hydroxyethyl starch | 0.028 | 0.171 |
| Ammonium acetate | 0.003 | Trace ~0.00003 |
| Histidine | 0.001 | 0.005 |
| Formulated mRNA-LNP | 0.027 | 0.164 |
| Ultrapure water | Quantity sufficient to | — |
| | target volume | |

### Example 3

### Formulation 3

In another example, a different formulation was tested for effects on polynucleotide complexes for use in spray-drying techniques. In this example, an aqueous solution of formulations illustrated in Table 3 were used and made according to the details in Example 1.

TABLE 3
Composition of Formulation 2 as a liquid and spray dried product

| Component | Liquid w/w | Spray dried w/w |
|---|---|---|
| Trehalose | 0.106 | 0.640 |
| Hydroxyethyl starch | 0.028 | 0.171 |
| Polyvinyl alcohol | 0.001 | 0.01 |
| Ammonium acetate | 0.003 | Trace ~0.00003 |
| Histidine | 0.001 | 0.005 |
| Formulated mRNA-LNP | 0.027 | 0.164 |
| Ultrapure water | Quantity sufficient to | — |
| | target volume | |

### Example 4

### Formulation 4

In another example, leucine was added to polynucleotide complexes for use in spray-drying techniques was tested. In this example, an aqueous solution of formulations illustrated in Table 4 was made according to the details in Example 1.

TABLE 4
Composition of Formulation 4 as a liquid and spray dried product

| Component | Liquid w/w | Spray dried w/w |
|---|---|---|
| Trehalose | 0.106 | 0.640 |
| Hydroxyethyl starch | 0.028 | 0.171 |
| Leucine | 0.03 | 0.15 |
| Histidine | 0.001 | 0.005 |
| Ammonium acetate | 0.003 | Trace ~0.00003 |
| mRNA-LNP | 0.027 | 0.045 |
| Ultrapure water | Quantity sufficient to | — |
| | target volume | |

### Example 5

### Spray-Drying

In another exemplary method, polynucleotide-containing and control materials were spray-dried as previously disclosed, with standard setpoint ranges according to a procedure document for the spray dryer (e.g., Buchi B-290 spray dryer). Standard setpoint ranges are detailed in Table 4 below. The batches were completed as expected and had results that fell in the average range.

TABLE 5

| Inlet | Drying Gas | Atomizing Gas | Feed |
|---|---|---|---|
| Temperature | Flow Rate | Flow Rate | Flow Rate |
| (° C.) | (m³/min) | (L/min) | (mL/min) |
| 90-130 | 0.4-0.62 | 5-10 | 0.5-2.0 |

### Example 6

### Analytical Methods

### Characterize Spray Dried Powders (Microparticles)

Analytical characterization was performed on the spray dried powders (microparticles) produced in Examples 1-4. In certain methods, the Malvern Mastersizer 2000 was and can be used to determine particle size distribution to assess features of these processes.

In another exemplary method, moisture content was measured using, for example, Karl Fisher titrations using a Metrohm Thermoprep unit. Moisture content is important when assessing stability of an essentially dried formulation containing at least one polynucleotide complex for storage and especially in prolonged storage to reduce degradation and/or contamination of a stabilized construct. Reduced moisture leads to more stable powders (microparticles) for transport, storage, reduced cold storage requirements and the like.

An example of these results for each batch are outlined in Table 6 below.

TABLE 6
Spray dried powder characteristics of Formulation 1

| | | Average Particle | Moisture Content |
|---|---|---|---|
| Sample | API | Size (um) | (wt %) |
| Example | Placebo | 7.92 | 2.26 |
| 1 - Lot 1 | | | |
| Example | LNP | 7.36 | 1.57 |
| 1 - Lot 2 | | | |
| Example | Placebo | 8.08 | 2.91 |
| 2 - Lot 1 | | | |
| Example | LNP | 8.64 | 2.35 |
| 2 - Lot 2 | | | |
| Example | Placebo | 11.38 | 3.42 |
| 3 - Lot 1 | | | |
| Example | LNP | 8.42 | 1.71 |
| 3 - Lot 2 | | | |
| Example | Placebo | 5.68 | 1.60 |
| 4 Lot 1 | | | |
| Example | LNP | 5.78 | 1.00 |
| 4 - Lot 2 | | | |

In these exemplary methods, these formulation batches resulted in powders with acceptable properties and yield. In certain examples, these formulated powders can be used to further apply at least one coating or encapsulation layer for additional stability or to provide a time-delivery layer. In some methods, these formulated powders can be used to further apply at least one coating or encapsulation layer for providing an adjuvanted coating or encapsulation to induce an immune response, if desired (e.g., alum coated).

### Example 7

### Cell-Based Assay

In another exemplary method, cells (e.g., HeLa cells) having a passage of lower than 20 are seeded in 96-well plate format. About 18 hours after seeding, cells are transfected with aqueous stock mRNA LNP or spray-dried mRNA LNP diluted in a media (e.g., complete media, MEM +10% FBS) in a concentration ranging from about 0.1-100 ng mRNA per well. The mRNA present in these formulations encodes a reporter fluorescent protein (e.g., GreenLantern). Media is replaced about 4 hours after transfection. Expression peak of the mRNA is achieved about 24 hours after transfection of the cells and is read using a Plate Reader (Green Cube gain 50) in PBS or using scanning confocal. These protocols can be used to assess viability of any targeted polynucleotide or polynucleotide complex contemplated herein compared to aqueous starting material. In addition, protein expression and/or immunogenicity of a polynucleotide contemplated herein can be tested on an antigen-encoding polynucleotide construct in vivo and/or in vitro.

An example of cell-based assay result for formulation 1-4 is outlined in Table 7 below.

TABLE 7
mRNA expression on Spray Drying Formulations
1-4 relative to aqueous starting material.

| Formulation | mRNA Expression (%) |
|---|---|
| 1 | 15 |
| 2 | 15 |
| 3 | 34 |
| 4 | 1 |

All of the compositions and methods disclosed and claimed herein can be made and executed without undue experimentation in light of the present disclosure. While the compositions and methods have been described in terms of embodiments, it is apparent to those of skill in the art that variations maybe applied to the compositions and methods and in the steps or in the sequence of steps of the methods described herein without departing from the concept, spirit and scope herein. More specifically, certain agents that are both chemically and physiologically related may be substituted for the agents described herein while the same or similar results would be achieved. All such similar substitutes and modifications apparent to those skilled in the art are deemed to be within the spirit, scope and concept as defined by the appended claims.

What is claimed is:

1. An aqueous composition comprising:
a polynucleotide construct or complex thereof or modified polynucleotide construct or complex thereof; or a polynucleotide construct or complex thereof or modified polynucleotide construct or complex thereof encoding one or more therapeutic agents, and one or more non-reducing disaccharide agents; and one or more aqueous solutions, wherein the aqueous composition does not include an unrelated adjuvant wherein an unrelated agent comprises an adjuvant capable of inducing a general immune response in a subject.

2. The composition according to claim 1, further comprising a single lipid nanoparticle (LNP) or a mixture of LNPs.

3. The composition according to claim 1, wherein the one or more non-reducing disaccharide is selected from the group consisting of trehalose, sucrose, lactose, and a combination thereof or combinations thereof.

4. The composition according to claim 1, wherein the one or more aqueous solutions comprises comprise histidine, H₂O, or a combination thereof.

5. (canceled)

6. The composition according to claim 1, further comprising at least one additional agent, wherein the at least one additional agent comprises one or more of hydroxypropyl-beta-cyclodextrin, lysine, leucine, silk fibroin, inulin, alternative sugars comprising one or more of dextrose, maltodextrin, sorbitol, mannitol, dextran, maltotriose and a combination thereof.

7. The composition according to claim 1, further comprising at least one additional agent, wherein the at least one additional agent comprises one or more agent capable of at least one of, preserving LNP integrity, and reduce or prevent LNP leaking.

8. The composition according to claim 1, wherein the composition has minimal to no Tris buffer.

9. The composition according to claim 1, further comprising one or more nonionic starch derivative, other starch, or polysaccharide, or combination thereof.

10. The composition according to claim 9, wherein the one or more nonionic starch derivative comprises one or more of hydroxyethyl starch, succinylated gelatin, and a derivative thereof.

11. (canceled)

12. The composition according to claim 1, further comprising one or more polymers, wherein one or more polymers comprises one or more of: polyvinyl alcohol (PVA), polyacrylic acid (PAA), polyethylene glycol (PEG), polymethacrylate-based copolymers (e.g., Eudragit), povidone or other high molecular weight polymer or other low solubility polymer, or combination thereof.

13. The composition according to claim 1, further comprising one or more polymers, wherein the polymer comprises PVA.

14. (canceled)

15. The composition according to claim 141, further comprising at least one cellulose agent, wherein the at least one cellulose agent comprises at least one of low molecular weight carboxymethyl cellulose (CMC) medium molecular weight CMC, high molecular weight CMC, chitosan, pectin, dextran, or a combination thereof.

16. The composition according to claim 1, wherein the polynucleotide construct or complex thereof or modified polynucleotide construct or complex thereof; or the polynucleotide construct or complex thereof or modified polynucleotide construct or complex thereof encoding one or more therapeutic agents, comprises any form of one or more of DNA, RNA, mRNA, or a mixture or complex thereof;
and when present, the therapeutic agents comprise one or more antigens or immunogenic agent; or includes at least one polynucleotide capable of inducing an immune response in a subject or has immunomodulatory properties.

17. (canceled)

18. The composition according to claim 1, wherein the polynucleotide construct or complex thereof or modified polynucleotide construct or complex thereof; or the polynucleotide construct or complex thereof or modified polynucleotide construct or complex thereof encoding one or more therapeutic agents comprises at least one antigen specific to one or more of a pathogenic virus, a pathogenic bacteria, a fungal pathogen; a chimera, a toxin, or a tumor antigen or a combination thereof.

19. The composition according to claim 1, further comprising one or more additional volatilizing agents, comprising an alcohol; optionally, wherein the alcohol comprises methanol or ethanol.

20. The composition according to claim 1, further comprising one or more volatile salts; optionally, wherein the one or more volatile salts comprise one or more of ammonium acetate, ammonium formate, ammonium carbamate, ammonium carbonate, ammonium bicarbonate, triethylammonium acetate, triethylammonium formate, triethylammonium carbonate, trimethylamine acetate trimethylamine formate, trimethylamine carbonate, pyridinal acetate and pyridinal formate, or combinations thereof.

21. (canceled)

22. The composition according to claim 1, wherein the polynucleotide construct or complex thereof or modified polynucleotide construct or complex thereof; or the polynucleotide construct or complex thereof or modified polynucleotide construct or complex thereof encoding one or more therapeutic agents comprises at least one polynucleotide encoding a recombinant polypeptide or protein; a virus-like particle; a live virus; a live, an attenuated virus; an inactivated virus; a toxoid; or fragment thereof or a combination thereof.

23. The composition according to claim 1, wherein the polynucleotide construct or complex thereof or modified polynucleotide construct or complex thereof; or the polynucleotide construct or complex thereof or modified polynucleotide construct or complex thereof encoding one or more therapeutic agents comprises at least one polynucleotide encoding at least one of, or antigen derived from: human papilloma virus (HPV), lentivirus, corona virus, retrovirus, human deficiency virus, Ebola virus, poliovirus, norovirus, rotavirus, hepatitis A, hepatitis, B, hepatitis C, dengue virus, varicella-zoster virus, herpes simplex virus, cytomegalovirus, Japanese encephalitis virus, West Nile virus, Zika virus, Haemophilus influenzae type b, measles virus, mumps virus, rubella virus, respiratory syncytial virus, influenza virus, yellow fever virus, rabies virus, smallpox virus, parvovirus, chikungunya virus, Corynebacterium diptheriae, Clostridium tetani, Clostridium botulinum, Bordetella pertussis, Streptococcus pneumoniae, Neisseria meningitides, Salmonella spp., Bacillus anthracis, Yersinia spp., or a combination thereof.

24. The composition according to claim 1, wherein the polynucleotide construct or complex thereof or modified polynucleotide construct or complex thereof; or the polynucleotide construct or complex thereof or modified polynucleotide construct or complex thereof encoding one or more therapeutic agents comprises at least one polynucleotide encoding at least one of, or antigen derived from: canine parvovirus, canine distemper virus, canine adenovirus, rabies virus, canine parainfluenza virus, canine influenza virus, canine corona virus, measles virus, Bordetella bronchiseptica, Leptospira spp., Borrelia burgdorferi, feline herpesvirus 1, feline calicivirus, feline panleukopenia virus, feline leukemia virus, feline immunodeficiency virus, virulent systemic feline calicivirus, Chlamydophila felis, Pasteurella haemolytica, Eastern equine encephalomyelitis virus (EEEV), Western equine encephalomyelitis virus (WEEV), Venezuelan equine encephalomyelitis virus (VEEV), Chikungunya virus, West Nile virus, equine influenza virus, equine herpesvirus, Streptococcus equi equi, Clostridium tetani, Neorickettsia risticii, bovine herpesvirus, parainfluenza type 3 virus, bovine viral diarrhea virus, bovine respiratory syncytial virus, Clostridium chauvoei, Clostridium septicum, Clostridium novyi, Clostridium perfringens type C, Clostridium perfringens type D, Clostridium haemolyticum, Marek's disease virus, reovirus, avian encephalomyelitis virus, avian influenza virus, avipoxviruses, chicken anemia virus, Pasteurella multocida, Newcastle disease virus, Riemerella anatipestifer, duck herpesvirus 1, duck hepatitis virus, Cryptococcus spp., Aspergillus spp., Blastomyces spp., Candida albicans, Paracoccidioides spp., Sporothrix spp., Histoplasma capsulatum, Pneumocystis jirovecii, Coccidioides immitis, or a combination thereof.

25-29. (canceled)

30. The composition according to claim 1, wherein the composition is essentially dry.

31. (canceled)

32. The composition according to claim 30, wherein the essentially dry composition further comprises at least one partial or complete coating layer for encapsulating the essentially dry composition comprising wherein the complete coating or encapsulation comprises an atomic layer deposition (ALD) applied coating or encapsulation of the essentially dry composition.

33. The composition according to claim 32, wherein each layer of the at least one partial or complete coating layer comprises one or more coating layers of metallo-organic-, metal oxides-, metal alkoxides-, and/or aluminum-based material.

34. The composition according to claim 32, wherein each layer of the at least one partial or complete coating layer comprises one or more of aluminum oxide (Al₂O₃), aluminum alkoxide, silicon dioxide (SiO₂), zinc dioxide (ZnO₂), titanium dioxide (TiO₂), and silicon nitride (Si₃N₄) or combinations thereof.

35. The composition according to claim 32, wherein the essentially dry composition or partially or completely coated or encapsulated essentially dry composition is reconstituted.

36. A pharmaceutical composition comprising the reconstituted composition according to claim 35, and a pharmaceutically acceptable excipient.

37. A method of preparing a composition according to claim 1, the method comprising:
combining the components according to claim 1 to create a liquid formulation; and at least one of: atomizing the liquid formulation in a gas stream to create particulates, and spray-drying the liquid formulation to create an essentially dry formulation of particulates and/or microparticles.

38-40. (canceled)

41. The method according to claim 37, wherein the particulates and/or microparticles further comprise at least one complete coating for encapsulating the particulates or the microparticles comprising the polynucleotide construct or complex thereof or modified polynucleotide construct or complex thereof; or the polynucleotide construct or complex thereof or modified polynucleotide construct or complex thereof encoding the one or more therapeutic agents.

42-44. (canceled)

45. The method according to claim 37, wherein drying gas used for the spray-drying has a relative humidity of less than 50%, or less than 40%, or less than 30%, or less than 20%, or less than 10% or less than 5%.

46. The method according to claim 37, wherein drying gas used for the spray-drying process has an inlet gas or nozzle temperature of less than 200° C., or less than 190° C., or less than 180° C., or less than 170° C., or less than 160° C., or less than 150° C., or less than 140° C., or less than 130° C., or less than 120° C., or less than 110° C., or less than 100° C., or less than 90° C., or less than 80° C., or less than 70° C., or less than 60° C., or 50° C. or less.

47. The method according to claim 37, wherein the liquid formulation further comprises at least one alcohol agent and permitting a lower drying temperature comprising a temperature of 200.0° C. or less for spray-drying the particulates or liquid formulation.

48. The method according to claim 37, wherein drying gas flow rates for the spray-drying comprise about 0.01 to about 5.0 m3/min, or about 0.1 to about 2.5 m3/min, or about 0.1 to about 1.5 m3/min, or about 0.1-1.2 m3/min.

49. The method according to claim 37, wherein atomizing gas flow rates comprise about 0.01 to about 100 L/min, or about 0.05 to about 75 L/min, or about 0.01 to about 75 L/min, or about 0.1 to about 50 L/min.

50. The method according to claim 37, wherein feed flow rates for the spray-drying comprise about 0.01 to about 50 g/min, about 0.05 to about 40 g/min, or about 0.1-35 g/min.

51. The method according to claim 37, further comprising using cyclone gas injected below the spray drying chamber for separating the particulates or the essentially dry formulation, and wherein cyclone gas flow rates comprise about 1.0 to about 1,000 L/min, about 1.0 to about 750.0 L/min, about 1.0 to about 500.0 L/min, or about 1.0-400.0 L/min.

52. The method according to claim 37, wherein drying gas for the spray-drying comprises at least one of ambient air, dehumidified air, or dry nitrogen, or any combination thereof.

53-54. (canceled)

55. A method for eliciting an immune response against one or more pathogenic organisms or stimulate an immune response against cancer or other antigens in a subject, the method comprising administering to the subject a pharmaceutical composition according to claim 36, wherein the polynucleotide construct or complex thereof or modified polynucleotide construct or complex thereof; or the polynucleotide construct or complex thereof or modified polynucleotide construct or complex thereof encoding one or more therapeutic agents encodes one or more immunogenic agent derived from at least one pathogenic organism, cancer or other antigen, and eliciting an immune response in the subject to the one or more therapeutic agents.

56-57. (canceled)

58. A kit comprising at least one composition according to claim 1, and at least one container.
